(19) United States
(12) Patent Application Publication
(10) Pub. No.: US 2022/0114491 A1
(43) Pub. Date: Apr. 14, 2022

(54) ANONYMOUS TRAINING OF A LEARNING MODEL

(71) Applicant: AquaSys LLC, Washington, DC (US)

(72) Inventors: Kevin Kelly, Washington, DC (US); Calden Carroll, Washington, DC (US); Adam Koeppel, Washington, DC (US); Tyler Locke, Washington, DC (US)

(21) Appl. No.: 17/497,529

(22) Filed: Oct. 8, 2021

Related U.S. Application Data
(60) Provisional application No. 63/089,644, filed on Oct. 9, 2020.

(51) Int. Cl. G06N 20/00; G06N 3/04

(52) U.S. Cl. CPC G06N 20/00; G06N 3/0427

(57) ABSTRACT

Systems, methods, and programs for privately and securely providing accurate machine learning models to anonymous clients for various applications. Discrete model classes of models are trained on non-anonymous datasets at a centralized server and served to anonymous clients. Clients validate each model against its own localized datasets and retain the most accurate model. Clients improve their model locally through transfer learning on new datasets, and share the updated, anonymized parameters with a centralized computer. The centralized server aggregates and updates model parameters for each respective discrete model class. The improved models may be served to future and existing clients.

The embodiments described and recited herein pertain generally to improving models for different discrete model classes anonymously, and to automatically selecting best-fit models from the different model class of models for a given client.

### BACKGROUND

Typical machine learning algorithms are trained on a large dataset and are periodically improved through a process of transfer learning. A generic “one-size-fits-all” approach can provide a generic model to all clients, which will be gradually improved with localized, user-specific data over time through transfer learning. Alternatively, client-targeted models can be deployed that were trained on datasets very similar to those generated by the client. These models will provide more accurate predictions “out-of-the-box”, e.g., at installation and initial start-up, but at the expense of potential loss of privacy from the client to the service provider (e.g., age, gender, location, etc. related to the trained dataset must be shared with the service provider in order to narrow the model class of the model to be provided to the client).

### SUMMARY

In accordance with at least one example embodiment, a computing system for obtaining a trained model privately and securely includes: at least one processor; at least one data storage device; a neural network; and machine readable instructions stored in the data storage that when executed by the at least one processor causes the system to: define, in a cloud-based computing system, a plurality of discrete model classes that include a plurality of machine learning models; receive by the cloud-based computing system, at least one dataset for modeling the plurality of discrete model classes; train at least one respective machine learning model of the machine learning models for each model class of the plurality of discrete model classes using the at least one dataset using the neural network; transmit the plurality of trained learning models associated with each model class to at least one anonymous client; receive updated parameters from the at least one anonymous client, wherein the updated parameters are from a selected trained model by the at least one anonymous client; aggregate and update parameters of the plurality of machine learning models by the neural network; and transmit the updated plurality of machine learning models to at least one client.

In another example embodiment, the at least one anonymous client includes a processor enabled device that includes memory, a processor, and machine readable instructions stored in the memory that when executed by the processor causes the processor enabled device to: validate each one of the plurality of trained learning models using a localized dataset, select one of the plurality of trained learning models having the highest accuracy among the plurality of trained learning models, retrain the selected one of the plurality of trained learning models using new datasets obtained by the at least one anonymous client through transfer learning.

The various embodiments include at least one of and/or any combination of the following features:

the plurality of discrete model classes is subdivided into a plurality of submodel classes, wherein each submodel class of the plurality of submodel classes includes at least one machine learning model;

the system further including transmitting the at least one machine learning model from any submodel class of the plurality of submodel classes associated with the selected one of the plurality of trained learning models having the highest accuracy;

the processor enabled device of the anonymous client is further configured to: validate each one of the at least one machine learning model for the submodel class using a localized dataset of the at least one anonymous client; select one of the plurality of trained learning models from the submodel class having the highest accuracy; retrain the selected one of the plurality of trained learning models from the submodel class using new datasets obtained by the at least one anonymous client through transfer learning; transmit updated parameters used in the selected one of the plurality of trained models of the submodel class to the neural network;

the processor enabled device is further configured to delete any remaining trained learning model that was not selected as having the highest accuracy;

the system is further configured to: retransmit the plurality of trained learning models associated with each model class to the at least one anonymous client after a predetermined amount of time; and the at least one anonymous client is further configured to validate each one of the plurality of trained learning models using the localized dataset of the at least one anonymous client; select one of the plurality of trained learning models having the highest accuracy; train the selected one of the plurality of trained learning models using new datasets obtained by the at least one anonymous client through transfer learning; transmit the updated parameters used in the selected one of the plurality of trained models to the neural network;

the predetermined amount of time is every thirty days or monthly;

the at least one anonymous client is configured to transmit any updated parameters used in the selected one of the plurality of trained models to the cloud-based computing system after a predetermined amount of time;

the plurality of discrete model classes is directed to soil mapping and the at least one data set includes data from representative soil types;

the plurality of machine learning models includes machine learning models for each representative soil type;

wherein the localized dataset includes data obtained from a plurality of sensors installed at a location of the at least one anonymous client, and wherein the plurality of sensors collect data that includes at least one of soil volumetric moisture, soil capacitance, soil tension, soil temperature, air humidity, air temperature, and barometric pressure;

further including: downloading a soil-specific machine learning model from the updated plurality of machine learning models for a soil type of the at least one client, and wherein the updated plurality of machine learning models are soil-specific models for each model class of the plurality of discrete model classes; and

the plurality of discrete model classes is directed to predictive text for regional dialects and the at least one data set includes data from different regions that speak the dialect.

In accordance with at least another example embodiment of a method for obtaining a trained model privately and securely described and recited herein, the method including: defining, in a cloud-based computing system, a plurality of discrete model classes, the plurality of discrete model classes comprising a plurality of machine learning models; receiving, by the cloud-based computing system, at least one dataset for modeling the plurality of discrete model classes; training the plurality of machine learning models for each model class of the plurality of discrete model classes using the at least one dataset using a neural network; transmitting the plurality of trained learning models associated with each model class to at least one anonymous client; validating each one of the plurality of trained learning models by the at least one anonymous client using a localized dataset of the at least one anonymous client; selecting one of the plurality of trained learning models having the highest accuracy; retraining the selected one of the plurality of trained learning models using new datasets obtained by the at least one anonymous client through transfer learning; transmitting updated parameters used in the selected one of the plurality of trained models to the neural network; aggregating and updating parameters of the plurality of machine learning models by the neural network; and transmitting the updated plurality of machine learning models to at least one client.

### BRIEF DESCRIPTION OF THE DRAWINGS

In the detailed description that follows, embodiments are described as illustrations only since various changes and modifications will become apparent to those skilled in the art from the following detailed description. The use of the same reference numbers in different figures indicates similar or identical items.

FIG. 1 is a schematic representation of a computing system in accordance with at least one example embodiment.

FIG. 2 is a schematic diagram of a neural network used in the computing system in accordance with at least one example embodiment.

FIG. 3 is schematic diagram of decision process for model validation and selection in accordance with at least one example embodiment.

FIG. 4 is a schematic diagram illustrating the use of submodels in accordance with at least one example embodiment.

FIG. 5 is a schematic representation of a plurality of discrete model classes based on the soil texture pyramid and model selection in accordance with a least one example embodiment.

FIG. 6 is a schematic representation of a computing system in accordance with at least another example embodiment.

FIG. 7 is a schematic representation of a plurality of discrete model classes based on the regional dialects and model selection in accordance with a least one example embodiment.

FIG. 8 is workflow diagram for obtaining the machine learning model in accordance with at least one example embodiment.

### DETAILED DESCRIPTION

In the following detailed description, reference is made to the accompanying drawings, which form a part of the description. In the drawings, similar symbols typically identify similar components, unless context dictates otherwise. Furthermore, unless otherwise noted, the description of each successive drawing may reference features from one or more of the previous drawings to provide clearer context and a more substantive explanation of the current example embodiment. Still, the example embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein and illustrated in the drawings, may be arranged, substituted, combined, separated, and designed in a wide variety of different configurations, all of which are explicitly contemplated herein.

Typically, machine learning models may be run locally by a processor enabled device of a specific client or run on a cloud-based processing system. It is appreciated that a client is a user of a machine learning model who receives as output a prediction when data is input into the machine learning model. The client may be an organization or group or individual that has a request for a plurality of machine learning models to solve a common problem set. Cloud-based processing of machine learning models has many benefits as compared to training machine learning models locally. For example, the cloud-based approach utilizes a processing system that includes a plurality of computing devices and processors that train a neural network to create a machine learning (ML) model for a variety of applications. Since the training of the neural network to create an ML model is generally computationally intensive, the cloud-based approach provides the computational resources for generating the machine learning models that are not typically available when training machine learning models locally.

Since the training of the ML model may require large amounts of data, all of the data necessary for the training of the ML model may not be available for the proper training on the cloud-based processing system since one or more clients may not want to provide corresponding private data unless the client is assured that the data remains anonymous, e.g., private. That is since the data transferred to the cloud-based processing system may not be sufficiently secured to maintain the privacy of the data, a client may be less likely to help in the training of global machine learning models. Additionally, while a generic global machine learning model may be usable by at least some clients, such generic global machine learned model may not provide a high level of accuracy for specific clients. For example, if the data used to train the machine learning model is generic, the resulting generic machine learning model might not include any site specific conditions that would affect the prediction of the machine learning model. Although the generic machine learned model may be improved by a transfer learning process by a localized client, since the generic machine learned model was not trained on data that is specific for the localized client, the training of the generic machine learning model may take too much time to achieve a high-level of accuracy and may not be available for “out of the box” use, e.g., make accurate predictions after receipt of the generic machine learned model. That is, the global machine learned model that is trained using generic data would need to be initialized by the localized client using site specific data, which may take up to a month, six months, or longer to collect the data, to receive the necessary parameters and weights to make accurate predictions, before the machine learned model could be used to make the predictions.

Machine learning models that are trained locally also have some benefits to a client as compared to the cloud-based processing system. For example, the local training of a machine learning model that uses a local processing system, e.g., computer, tablet, phone, other devices having a processor, etc., has access to data that is more secure than cloud-based processing. The data, for example, is not uploaded to the cloud but provided locally in a database or is only accessible by the client, e.g., password encrypted on a web-accessible database. Such local processing, however, does not have the computational efficiency or resources available as in the cloud-based processing system. Additionally, a model trained based on local data might not include training data that may be useful for future predictions since the local data might not include data from other clients that have similar conditions to provide a more robust trained model. Machine learning models that are run locally may also provide more accurate predictions “out-of-the-box”, e.g., on initial installation and start-up, than a generic global machine learned model from a cloud-based processing system, since the local machine learning models include client-targeted and specific models that were trained on datasets that may be very similar to another client. While such out-of-the box models likely provide more accurate predictions, these models may have the potential for loss of privacy of the underlying data used to train the model, since certain parameters of the client data may be shared to narrow the model class in which the model is to be used, e.g., match the datasets using, for example, age, gender, location, etc. to provide the most accurate model.

That is, while data may be useful for planning and optimization of any system or organization at different stages, the use of localized data from specific clients may also allow a harmful invasion of privacy which may lead to abuses in using such underlying information. In order to optimize a system or organization, a balance must be struck between data sharing and data privacy.

In an embodiment, a system, method, and program stored on non-transitory computer readable media are provided for planning and optimization of machine learning models that has the benefits of using shared data, while maintaining data privacy and security of the shared data by not revealing or sharing the underlying data, e.g., the source data, that was used for training specific machine learning models. Other advantages are discussed herein, for example, the enhanced operation of machine learning systems in areas in which access to high speed data networks is not available.

In an embodiment, the systems, methods, and programs provide for automatic selection of the best-fit model for a given client in which higher-performance models are provided privately, securely, and iteratively to improve the models in a distributed and anonymous manner. For example, in an embodiment, the computing system includes at least one processor, at least one data storage device, a neural network, and machine readable instructions, which when executed by the processor controls the system to define, in a cloud-based computing system, a plurality or an array of discrete model classes for a particular problem set, in which each model class or array includes at least one machine learning model. The system is also configured to receive representative datasets for training neural networks to provide a machine learning model for each underlying model class and/or function.

In another embodiment, each model class may be further subdivided into submodel classes as needed by the client, group, or organization, etc., in which each submodel class includes its own representative machine learning model. The machine learning model(s) for each top-level model class model (and/or any submodel classes) are transmitted to at least one anonymous client. The anonymous client validates each machine learning model using a supervised and localized dataset, and the accuracies of each model are determined. The machine learning model with the highest accuracy is selected and the remaining machine learning models may be deleted from memory. The anonymous client may transmit the model selection to the cloud-based computing system. The anonymous client uses the selected machine learning model to make predictions for the purposes of the application. The anonymous client updates the machine learning model(s) locally through transfer learning with the localized data to produce parameters for the local machine learning model. The parameters of the local machine learning model are associated with the corresponding model class and uploaded/transmitted to the cloud-based computing system anonymously. The cloud-based computing system may then aggregate the updated model parameters by model class and/or update the machine learning model of each categorical model. The cloud-based computing system may then transmit the updated machine learning models to all new and existing clients and the process iterates periodically and throughout the lifetime of the application.

It is appreciated that if the selected model class has associated submodel classes, the next tier of submodels may be transmitted to the anonymous client for validation and/or training, as well. The validation/training and analysis is repeated for the submodels until the best performing submodel is selected. The updated parameters of the selected submodel may then be transmitted to the cloud-based computing system. The updated parameters of the submodel may be used to update the selected submodel for the model class and/or any higher-level machine learning model parameter for the associated model class, e.g., top-level.

Further embodiments of the systems, methods, and programs for training a model privately, securely, and iteratively to improve the models in a distributed and anonymous manner, in which the best-fit model may be automatically selected for a given client, are discussed further below.

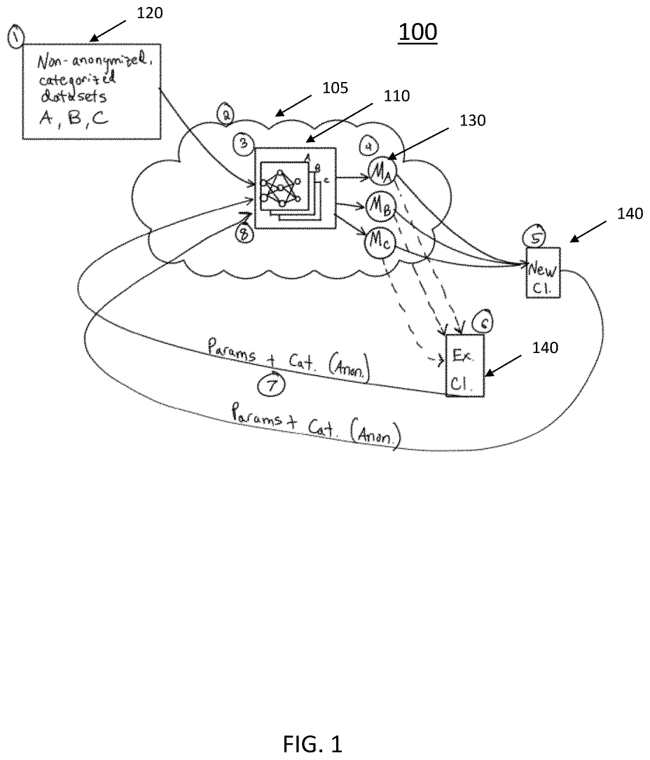

FIG. 1 is a schematic diagram of a computing system that includes neural networks for training a plurality of machine learning models privately and securely in accordance with at least some embodiments described and recited herein. As depicted in FIG. 1, the computing system 100 includes a cloud-based computing system 105 that includes a processing system that includes, for example, a distributed hardware system, a plurality of networked computing devices, data storage devices, and processors having memory that may be designed, programmed, or otherwise configured to train machine learning models using a neural network, e.g., a recurrent neural network (RNN). In an embodiment, the RNN can be a multivariate “many-to-one” or “many-to-many” or a “one-to-many” Long-Short-Term-Memory (LSTM) architecture that is used for the training of ML models, for example, soil type prediction, future water demand for a region, future pesticide demand and pest emergence, forecasting yield of a crop type, forecasting damage to a crop type due to adverse weather, backcast seasonal weather to determine what contributed to crop yield, and other agronomic, scientific or logical applications, and for predictive text, based on a target dataset and at least one of or some combination of feature datasets.

For example, the cloud-based computing system 105 includes memory and/or at least one data storage device having machine readable instructions which when executed controls the system to define a plurality of discrete model classes 110 related to particular problem sets, receive and/or access a plurality of datasets 120 related to the plurality of discrete model classes, and train a plurality of global machine learning models 130 using the plurality of datasets 120 by the neural network, for example, the RNN. In an embodiment, the problem to be solved may be provided by an organization, group, or client that has a particular problem that may be modeled given a particular data set. The organization, group, or client may access the cloud-based computing system 105 via the Internet, Intranet, or other way to access the distributed hardware system, the plurality of networked computing devices, etc.

The plurality of discrete model classes 110 are particular problem sets to be solved, in which each of the plurality discrete model classes may relate to the same particular problem, relate to different problems to be solved, and/or combinations thereof. The plurality of discrete model classes 110 includes the machine learning models 130 to solve the particular problem and may be top-level machine learning models related to the model class. For example, in an embodiment, the particular problem to be solved may be determining the type of soil provided at a client site and determining/executing certain actions in view of the type of soil, e.g., clay, sand, silt, loam, etc., and combinations thereof. In another embodiment, the particular problem to be solved may be providing predictive text based on the dialect of the client, facial recognition, etc.

Each of the plurality of discrete model classes may also each include submodel classes related to the respective top-level discrete model classes, in which each submodel class also includes a machine learning model, e.g., submodels for determining the soil conditions, e.g., wet, very wet, dry, very dry, etc.

Each data in the datasets 120 is global data that that has been collected and/or provided that may directly or indirectly influence the particular problems identified for the plurality of discrete model classes. For example, the dataset might include historical sensor readings, weather forecasts, and the predictions. Specifically, the plurality of datasets 120 are non-anonymous datasets that may be general/public data, provided by certain clients, groups of clients, organizations, etc., or any combination thereof related to the particular problem to be solved for the plurality of discrete model classes 110. The plurality of datasets 120 may include data, for example, related to general types of soil, e.g., clay, sand, silt, loam, combinations thereof, etc. and different conditions, e.g., wet, dry, very dry, very wet, ideal, cold, hot, etc. For example, the datasets may include soil temperature data, soil volumetric data, soil matric potential data, soil wetness data, soil density data, air humidity sensor, air temperature sensor, a barometric pressure, recorded precipitation, dew point, UV Index, solar radiation, cloud cover percentage, etc.

The plurality of machine learning models 130 are models trained using the plurality of datasets 120 by the neural network in which the machine learning model may be a relatively universal machine learning model that is an approximation of an underlying physical or natural system, based on the inherent physics of the system. The plurality of machine learning models 130 include machine learning models for each model class of the plurality of discrete model classes 110, e.g., creates global models for each top-level model class, and may further include machine learning models for any submodel classes until all or most discrete model classes and/or submodel classes are defined by a machine learning model. For example, in an embodiment, a machine learning model, e.g., a top-level model, may be provided for each model class, e.g., type of soil condition, etc., silt, sand, clay, loam, and combinations thereof. Lower-level submodels may be provided for each of the submodel classes, for example, machine learning models for silt in wet conditions, silt in dry conditions, etc. Thus, the trained machine learning model outputs the type of soil and/or soil condition from the inputs provided from the plurality of datasets 120. The machine learning models may use a single layer, multiple layers, feedback/recurrent layers, etc.

A number of factors contribute to the machine learning model's overall efficacy. To be successful, a model needs to be both accurate and broadly applicable. Using the right architecture for the problem, optimizing the hyper parameters, and choosing the right “stopping point” during model training, all factor into how well the model performs. Similarly, having the right inputs, or features, has an outsized impact on model performance. To make the model accurate, the model is trained with only those feature datasets that contribute positively to its error rate. Thus, optimizing the model for a particular target output, means being selective about what information is fed into it during the training phase.

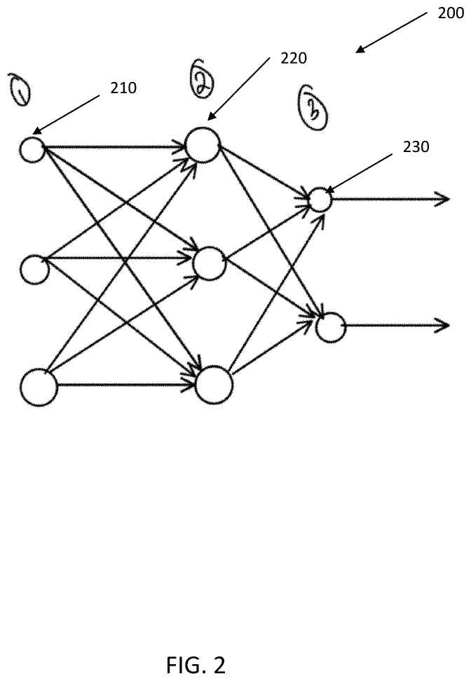

For example, as seen in FIG. 2, one way to determine if particular data from a dataset contributes positively to the model's performance may be by calculating the model's error. For example, the neural network has the global dataset 120 input at an input layer 210, and then through a plurality of hidden layers 220, determines what data from the global dataset 120 contributes positively to the machine learning model's performance by calculating the machine learning model's error. If a model has lower error (and therefore better performance) when a feature is included versus excluded, the dataset is deemed worthy of inclusion in the training phase. This process of adding and removing features is automated via a scripting language that iteratively compares the model performance with and without a particular feature, and can relatively quickly narrow the list of features for inclusion in the final training phase of model.

The modeling to the plurality of datasets may be considered complete and be used as a machine learning model, when the error of the modeling reaches a predetermined error rate, e.g., between 80%-99% accuracy or 1%-20% error threshold, and preferably between 90-95% accuracy or 5-10% error threshold. Error is calculated by comparing the model's accuracy against a known target dataset, with lower error being better. To make the model broadly applicable and not overfitted for one particular dataset, transfer learning may be used to adapt a model previously trained on a single dataset to generalize across several disparate datasets that can be unique to specific a geographic location, soil type, ambient environment, etc. The neural network then outputs the trained machine learning model at output layer 230.

Not only does the neural network include the output layer 230 that outputs a machine learning model for each model class, the neural network may also output what data is necessary for training the machine learning model for the specific model class and problem to be solved. For example, during the calculating the model's error, the feature data that contributes positively to the model error may be determined necessary for training. The data that is found to contribute positively to the machine learning model's performance may also be output at the output layer 230.

Referring back to FIG. 1, FIG. 1 also shows that the computing system includes at least one anonymous client 140 in which the computed-based computing system 105 is designed, programmed, or otherwise configured to transmit the plurality of machine learning models 130 for each model class of the plurality of discrete model classes 110 to at least one anonymous client 140 for further development and training. It is appreciated that the plurality of machine learning models 130 may be downloaded as a software application on a processor enabled device, e.g., computer, phone, tablet, microcontrollers, etc., of the at least one anonymous client 140 or may be accessible via web portal for download by the at least one anonymous client 140.

The at least one anonymous client 140 having the processor enabled device that includes memory, a processor, and machine readable instructions is designed, programmed, or otherwise configured to run and validate each of the plurality of machine learning models 130 for each model class of the plurality of discrete model classes 110 using data provided locally at the at least one anonymous client 140, e.g., local database. It is appreciated that the local databases may also include encrypted databases, e.g., AWS. That is, the local databases are datasets that are locally controlled by the anonymous client and not accessible by the cloud-based computing system. It is appreciated that the data collected locally relate to the feature data used to train the global machine learning model, e.g., the data may be collected for the same feature or input data inputted into the input layer of the neural network. For example, the local dataset might be data collected by a particular farmer using sensors for collecting agricultural conditions and weather, whereas, the global machine learning models are defined for a farmer co-op or national agricultural organization for the same data types.

In an embodiment, the data for the local databases is collected for a predetermined amount of time, e.g., 1 month or 30 days. It is appreciated that the predetermined amount of time may be any time length that allows the collection of data for training the machine learning model, e.g., one day, one week, one month, etc. The processor enabled device of the at least one anonymous client having the software program may be designed, programmed, or otherwise configured to separate the collected data into a test dataset and a validation dataset. The test dataset and the validation dataset may be separated based on the predetermined amount of time, e.g., when the predetermined amount of time is 1 month, the first three weeks may be used as the test dataset and the last week used as the validation dataset, or other arrangement that can be used for establishing a test dataset and a validation dataset.

Each of the machine learning models for each model class is run using the test dataset to produce a prediction. The machine learning models are then validated using the validation dataset to obtain the error for each machine learning model. After all of the plurality of machine learning models 130 are run by the at least one anonymous client 140, the machine learning model having the highest accuracy among the plurality of machine learning models based on the local dataset is selected, e.g., a machine learning model that has between 80-95% accuracy, whereas, the remaining models of the plurality of machine learning models has less than 80% accuracy. For example, if three machine learning models are used for three discrete model classes of soil types, e.g., clay, silt, and loam, the error may be 69%, 90% and 98%, respectively, for the three different discrete model classes of soil types. For example, the machine learning model that has the lowest Mean Absolute Error (MAE), most-closely describes the soil type at the given anonymous client site location and might be selected and retained for further training. However, the error is not intended to be limited to the MAE, but other representations of error, e.g., subtraction, standard deviation, standard error, relative error. It is appreciated that the remaining less accurate global machine learning models may then be discarded and/or deleted, e.g., removed by the at least one anonymous client 140.

After selection of the machine learning model with the lowest error, the at least one anonymous client 140 continues using the selected machine learning model to provide predictions and the selected machine learning model is retrained using data collected locally by the at least one anonymous client 140, e.g., using transfer learning, in which lower layers in the machine learning model are retrained with the local site specific data in which the local data that is collected is separated as a test dataset and validation dataset. The selected machine learning model is trained until the error of the modelling reaches a predetermined error rate, e.g., between 90%-99% accuracy or 1%-10% error threshold, and preferably between 95-99% accuracy or 1-5% error threshold. Error is calculated by comparing the model's accuracy against the local dataset, with lower error being better. It is appreciated that since the global machine learning model was reasonably accurate, e.g., between 80-95% accurate, when transmitted to the at least one anonymous client, less adjustments of the weighting parameters are needed to improve the accuracy of the selected machine learning model, e.g., compared to the original training of the global machine learning model. Thus, the local training by the at least one anonymous client is able to be performed with processor enabled devices that have less computing capacity than the neural network (or distributed network) since the training is less computationally intensive.

After the local machine learning model reaches the predetermine error rate, the resulting model parameters, e.g., weights, biases, etc., used in the local machine learning models are saved by the at least one anonymous client 140. The parameters of the local machine learning model may then be transmitted or uploaded to the cloud-based computing system periodically, e.g., once a week, once a month, etc. or opportunistically depending on parameter availability, network connectivity, battery state, etc. It is appreciated that other information may also be transmitted to the cloud-based computing system 100 to increase the reliability of the machine learning models, such as, test conditions, number of different data points, etc.

The cloud-based computing system 100 after receiving the updated parameters from the selected machine model, may be designed, programmed, or otherwise configured to aggregate all of the respective received parameters for the machine learning models of the plurality of machine learning models and update the parameters of the machine learning model for the respective model class. Periodically, e.g., once a week, once a month, etc., the cloud-based computing system 100 may update the parameters of the machine learning models received from the anonymous client(s), e.g., batch model update is performed in which the plurality of trained learning models is retransmitted to at least one anonymous client and the anonymous client repeats the validation, selection, training, and transmission of updated parameters of the selected machine learning model. In an embodiment, subsequent parameter updates from an anonymous client will overwrite an earlier update. In other embodiments, the parameters are aggregated, averaged, weighted, or otherwise computed from a plurality of anonymous clients for the different discrete model classes and machine learning models to maintain anonymity of the client. For example, in an embodiment, a weighted average may be used for an anonymous client using ten sensors for collecting data for the local dataset while another anonymous client only uses 2 sensors for collecting data for the local dataset in which the updated parameters from the anonymous client using ten sensors may have a higher weighted average when updating the parameters for the global machine learning models.

The cloud-based computing system 100 may then be designed, programmed, or otherwise configured to transmit the updated machine learning models to at least one other client to make predictions, e.g., not necessarily the anonymous client. It is understood that in an embodiment, the at least one other client has sensors or other data collection means to collect the same feature data used to train the updated machine learning model. For example, if the global machine learning model was trained with ten inputs or feature datasets, the at least one other client would have the same local inputs or feature data for running the updated machine learning model. That is, over time, the RNN can incorporate continuous transfer learning to tune to specific geographical locations of various clients to improve the accuracy of predictions. Thus, the global machine learning model for the respective model class, e.g., soil type silt, is improved for subsequent new clients by using the anonymous data that closest matches the subsequent client to obtain the most relevant parameters for the original global machine learning model, e.g., a model class or submodel class is modeled with data and characterized to match subsequent client conditions. It is appreciated that each of the global machine learning models may then be updated in similar manners using anonymous clients based on localized datasets. Since only the parameters of the models are transmitted by the at least one anonymous client and the parameters are aggregated by the cloud-based computing system 100, anonymity of the dataset is preserved. As a result, the subsequent new client may be able to forecast future conditions at a new (or similar) geographic location given the same dataset. Thus, it is appreciated that while the global machine learning models have greater accuracy than the local machine learning models for predicting for any condition or geographic location, the parameters and weights of the local machine learning model may be used to increase the accuracy of predictions for site specific or new geographic locations.

In an embodiment, the cloud-based computing system 100 may also output a machine learning model for a model class (or submodel class) only after a predetermined amount of parameter updates have been received. For example, after each of the parameters of the machine learning model have received two parameter updates, preferably five parameters updates, and most preferably ten parameter updates, the cloud-based computing system may transmit a specific model, e.g., for a selected model class, to a user client. The updated global machine learning model may replace the prior version(s) of the global machine learning model or be used to aggregate the parameters of the global machine learning model.

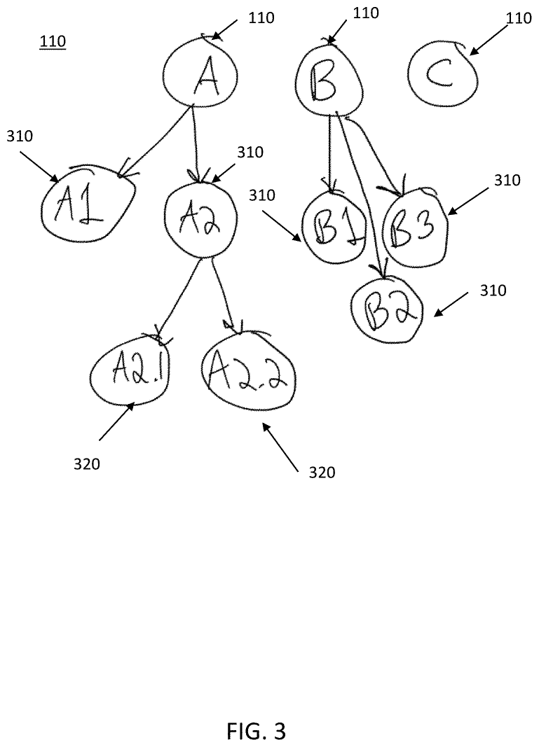

As illustrated in FIG. 3, in an embodiment of the invention, each model class 110 further includes a plurality of submodel classes 310 in which each submodel class includes submodels. It is appreciated that the submodels may also further include additional submodels 320 for further defining user specific conditions to provide the most accurate prediction for a specific client, e.g., using parameters derived from datasets that most closely matches the specific client. When the cloud-based computing system 105 includes the discrete model class and submodel classes, it is appreciated that the system is also configured to transmit any of the submodels from the submodel classes to the anonymous client 140 for local validation and training by the anonymous client. For example, the machine learning submodels may be related to determining whether the soil is dry, very dry, wet, very wet, etc. and the submodels are then trained using the localized data collected by the at least one anonymous client. Similar to the above process with respect to the top-level models, the anonymous client 140 selects one of the plurality of submodels having the highest accuracy and retains the selected one of the plurality of trained learning submodels. The selected trained learning submodel is then retrained using a new dataset obtained by the at least one anonymous client through transfer learning. Thereby, a local machine learning submodel may be obtained for specific conditions of the at least one anonymous client so that site specific recommendations for any subsequent client may be provided. The anonymous client 140 may then transmit the updated parameters for the trained submodel and the selection of the respective submodel class to the cloud-based computing system.

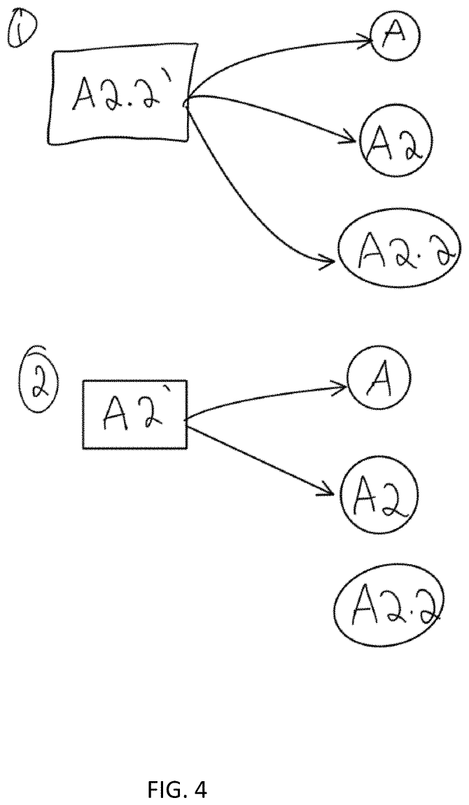

As seen in FIG. 4, it is appreciated that the parameters for the selected submodel(s) that are updated by the anonymous client 140 using the localized dataset may be used in a variety of ways. For example, the parameters from the updated submodel may be used to update parameters of the machine learning models at the cloud-based computing system 105 for any of the machine learning models of the associated submodel class or any higher level model class, e.g., any top-level models. The parameters of the submodel, however, are not typically used to update any lower level models, e.g., any child models of any sub-sub model classes.

It is appreciated that since only parameters or weights are being transmitted by the at least one anonymous client, minimal transmission bandwidth is required to send the parameters or weights to the cloud-based computing system. That is, since the full local machine learning model is not being transmitted, less internet bandwidth is necessary, and the anonymous client may continue running the local machine learning model to make the necessary predictions.

Further embodiments and examples are provided below.

In an exemplary embodiment, the computing system for obtaining a trained model privately and securely may be used for mapping soil types for different user clients. In this embodiment, an organization, group, company, or other organization that may have a plurality of user clients, establishes a problem to be solved and what discrete model classes are related to the problem to be solved. For example, the organization or group may be the National Future Farmers of America, National Farmers Union, American Farm Bureau Federation, American Farmland Trust, Institute of Food and Agricultural Sciences, Insurance agencies, Co-ops, etc. and the problem to be solved may be determining the soil type for a particular client. By determining the soil type of a particular client, the organization or group may then be able to provide the appropriate guidance and recommendations for the optimal growing conditions based on the soil type, e.g., irrigation intervals, seasonal growing, tilling, fertilization schedules, pesticides, etc.

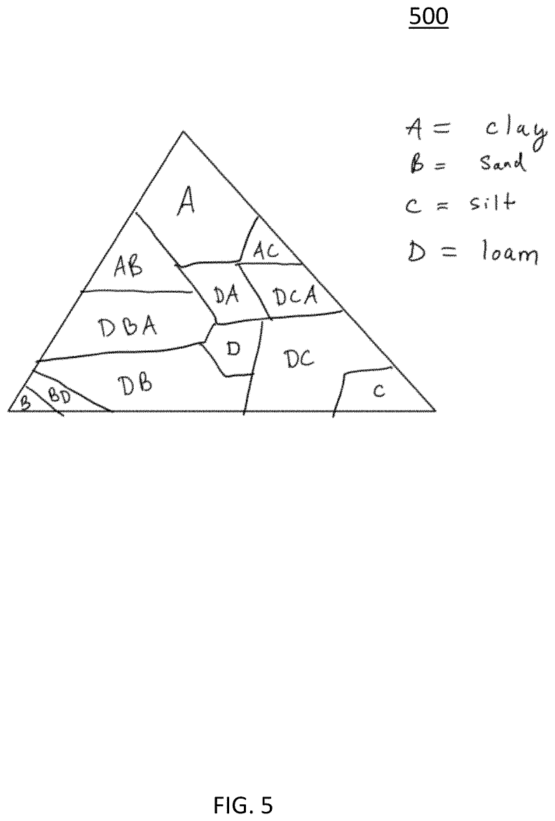

For example, FIG. 5 illustrates an example of the problem to be solved, in which the problem to be solved 500 is determining the different soil types at a client site and the plurality of discrete model classes that are associated with the different types of soil, such as, clay A, sand B, silt C, loam D, etc., and combinations thereof. It is appreciated that the soil texture pyramid may further include any associated submodel class, e.g., wet, very wet, dry, very dry, ideal, etc., that is associated with the top-level model class, e.g., clay, sand, silt, loam, etc.

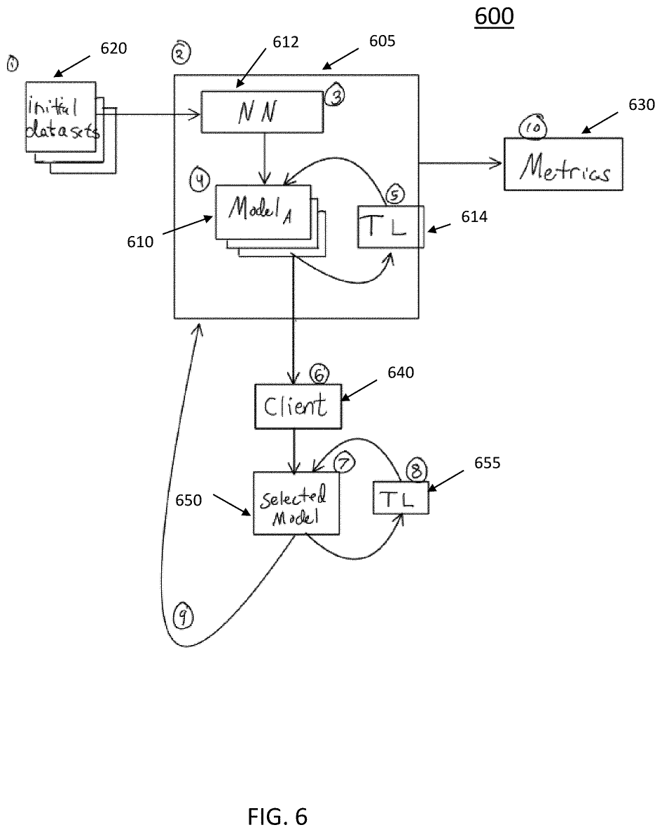

As seen in FIG. 6, the computing system 600 includes a cloud-based computing system 605, e.g., network of connected servers, networked computing devices, neural network, etc. The cloud-based computing system 605 includes neural networks 612, a plurality of discrete model classes 610 that includes a plurality of global machine learning models that uses a transfer learning process 614. The cloud-based computing system 605 may also include or is connected to an initial global dataset 620, a data base for storing feature metrics 630, and at least one anonymous client 640.

After the organization or group defines the problem to be solved, the cloud-based computing system 605 is designed, programmed, or otherwise configured to define the plurality of discrete model classes related to the problem to be solved, and determines the initial global machine learning models for each model class, e.g., determines initializing seed models that will be used by the user clients for each model class. It is appreciated that the initial global machine learning models may be a single model for each model class, e.g., a single model for each of clay, sand, silt, loam, etc., or a plurality of models and submodels for each model class, e.g., dry, very dry, wet, very wet, ideal, etc.

In determining the initial global machine learning models, the organization or group may upload and/or transmit a plurality of global datasets 620 related to the plurality of discrete model classes 610 to the cloud-based computing system 605. For example, the plurality of global datasets 620 may be obtained from experimental nodes that are installed in representative soil types for each model class and recorded across a multitude of soil states, e.g., ideal, very wet, very dry, cold, heat, etc. Multiple nodes may also be installed in each soil type to reduce the effective error of any one node's sensors. The sensors may be used to collect data, such as, soil temperature data, soil volumetric data, soil matric potential data, soil wetness data, soil density data, air humidity sensor, air temperature sensor, a barometric pressure, recorded precipitation, dew point, UV Index, solar radiation, cloud cover percentage, etc. It is appreciated that this global dataset is non-anonymous and may be public data, data collected by the organization or group, data provided from non-anonymous sources, data accessible from Internet sites, e.g., Weather.com, etc.

The cloud-based computing system 605 may then be designed, programmed, or otherwise configured to train the global machine learning models from the global dataset 620 using the neural network 612. The neural network 612 may be a recurrent neural network (RNN) in which the machine learning models are representative of each soil type for each model class, e.g., a machine learning model for clay, silt, loam, etc. A number of factors contribute to the machine learning model's overall efficacy. To be successful, a machine learning model needs to be both accurate and broadly applicable. To make the model accurate, the machine learning model is trained with only those feature datasets that contribute positively to its error rate. Thus, optimizing the machine learning model for a particular target output, means being selective about what information is fed into it during the training phase.

For example, the neural network 612 has the global dataset 620 input at an input layer, and then through a plurality of hidden layers, determines what data from the global dataset 620 contributes positively to the machine learning model's performance by calculating the machine learning model's error, e.g., obtains a set of metrics 630. If a machine learning model has lower error (and therefore better performance) when a feature is included versus excluded, the dataset is deemed worthy of inclusion in the training phase. This process of adding and removing features is automated via a scripting language that iteratively compares the machine learning model performance with and without a particular feature, and can relatively quickly narrow the list of features for inclusion in the final training phase of model.

The modeling to the plurality of global datasets may be considered complete and be used as a machine learning model, when the error of the modeling reaches a predetermined error rate, e.g., between 80%-99% accuracy or 1%-20% error threshold, and preferably between 90-95% accuracy or 5-10% error threshold. Error is calculated by comparing the model's accuracy against a known target dataset, with lower error being better. To make the model broadly applicable and not overfitted for one particular dataset, transfer learning algorithms 614 may be used to adapt a model previously trained on a single dataset to generalize across several disparate datasets that each can be unique in geographic location, soil type, ambient environment, etc.

In so doing, not only does the neural network output at the output layer a machine learning model for each model class 610, the cloud-based computing system 605 may also output the data that contributes positively to the model error and, thus, necessary for installation of sensors at a user client site to obtain such data, e.g., the set of metrics 630. That is, by using the global dataset, the neural network may be used to determine what features are necessary for the prediction of the machine learning model.

The organization or group may then use the cloud-based computing system 605 to transmit the plurality of global machine learning models for each model class 610 to at least one anonymous client 640, e.g., farmer, for further training and provisioning. For example, in an embodiment, a node including sensors for collecting data that was found to contribute positively to the machine learning model's training, e.g., the metrics 630, are installed at the site of the anonymous client 640. The sensors may include, for example, sensors for collecting soil temperature data, soil volumetric data, soil matric potential data, soil wetness data, soil density data, air humidity sensor, air temperature sensor, a barometric pressure, recorded precipitation, dew point, UV Index, solar radiation, cloud cover percentage, etc. It is appreciated that no initial information, e.g., soil type, is required to be known by the anonymous client 640 prior to installation of the node. The node may include a gateway, onboard memory, a processor, display, and an operation system. The plurality of global machine learning models for each model class 610 of the different soil types are saved on the node or other processor enabled device, e.g., computer, at the anonymous client site. The plurality of global machine learning models may be downloaded as software, through an application, etc. and saved at the local client site of the anonymous client 640.

The node collects data for a predetermined amount of time, e.g., 1 month or 30 days. It is appreciated that the predetermined amount of time may be any time length that allows the collection of data found useful for training of the machine learning model, e.g., one day, one week, one month, one year, etc. The node having the software program may then process the data by separating the data into a test dataset and a validation dataset. The test dataset and the validation dataset may be separated based on the predetermined amount of time, e.g., when the predetermined amount of time is 1 month, the first three weeks may be used as the test dataset and the last week used as the validation dataset, or other arrangement that can be used to establish a test dataset and a validation dataset.

The anonymous client 640 that has the processor enabled device that is designed, programmed, or otherwise configured to run each of the global machine learning models for each model class using the test dataset to produce a prediction, e.g., in which for N soil-specific models for each of the N discrete model classes, N predictions will be made. The global machine learning models are then validated using the validation dataset to obtain the error for each global machine learning model. For example, if three global machine learning models are used for three discrete model classes of soil types, e.g., clay, silt, and loam, the error may be 69%, 90% and 98%, respectively, for the three different discrete model classes of soil types. Thus, the global machine learning model that has the lowest error, most-closely describes the soil type at the given anonymous client site location and might be selected and retained for further training. It is appreciated that the remaining less accurate global machine learning models may then be discarded.

After selection of the global machine learning model with the lowest error, e.g., the model associated with the loam model class, the processor enabled device is designed, programmed, or otherwise configured to further trained the selected global machine learning model 650 using transfer learning algorithm 655, in which lower layers in the machine learning model 650 are retrained with data collected locally at the node. The selected global machine learning model 650 is trained until the error of the modelling reaches a predetermined error rate, e.g., between 90%-99% accuracy or 1%-10% error threshold, and preferably between 95-99% accuracy or 1-5% error threshold. It is also appreciated that any submodel class related to the selected model class, e.g., models related to the loam model class, may also be trained locally. For example, the machine learning submodels related to determining whether the soil is dry, very dry, wet, very wet, etc. are trained using the data collected by the node. Thereby, a local machine learning model may be obtained for the specific microclimate conditions and soil type of the at least one anonymous client 640 that is unique to the at least one anonymous client 640. Thus, the trained local machine learning model 650 may be used to predict the type of soil and condition of the soil, e.g., dry, in which the prediction is used to determine necessary actions and/or recommendations for improvement in agricultural conditions, e.g., increase irrigation, change irrigation schedules, increase/change fertilization, etc.

After the local machine learning model 650 reaches the predetermine error rate, the resulting model parameters, e.g., weights, biases, etc., used in the local machine learning models are saved in the node and/or processor-enabled device. The processor enabled device is designed, programmed, or otherwise configured to transmit the parameters of the local machine learning model to the cloud-based computing system 605 periodically, e.g., once a week, once a month, etc. or opportunistically depending on parameter availability, network connectivity, battery state, etc. It is appreciated that other information may also be transmitted to the cloud-based computing system 605 to increase the reliability of the global machine learning models. For example, the model class that was selected, e.g., loam model class, may be sent to the cloud-based computing system and/or any information that the anonymous client determines would be useful for the global machine learning model but which still maintains the anonymity of the anonymous client, e.g., a region, state, country of the anonymous client.

Periodically, e.g., once a week, once a month, etc., the cloud-based computing system 605 may be designed, programmed, or otherwise configured to update the parameters of the global machine learning models received from the anonymous client(s) 640, e.g., batch model update is performed. In an embodiment, subsequent parameter updates from an anonymous client 640 will overwrite an earlier update. In other embodiments, the parameters are aggregated, averaged, or otherwise computed from a plurality of anonymous clients 640 for the different discrete model classes and machine learning models to maintain anonymity of the client.

In an embodiment, the cloud-based computing system 605 may be designed, programmed, or otherwise configured to output a global machine learning model for a model class (or submodel class) only after a predetermined amount of parameter updates have been received. For example, after each of the parameters of the global machine learning model have received two parameter updates, preferably five parameters updates, and most preferably ten parameter updates, the cloud-based computing system will transmit a given soil-specific model, e.g., for a selected model class, to a user client. The updated global machine learning model may replace the prior version(s) of the global machine learning model or be used to aggregate the parameters of the global machine learning model.

The cloud-based computing system 605 may then be designed, programmed, or otherwise configured to transmit the updated global machine learning model(s) to new clients and/or existing clients that have the prior versions of the global machine learning model. It is appreciated that the global machine learning model has a high accuracy from initial installation at least because of the use of the transfer learning process for each soil-specific machine learning model for each model class (and submodel classes). That is, a new client or existing client may download the updated global machine learning model that is soil-specific for the new client or existing client, e.g., the global machine learning model(s) for the loam model class, so that a global machine learning model that most accurately represents the soil condition of the new client or existing client is selected, which is more accurate than previous models and does not require the time required to train a site specific model, e.g., shortcuts the training process for a site specific, e.g., microclimate, machine learning model. It is appreciated that the new client and/or existing client may be used for the continued training of the global machine learning models. Specifically, the new client and/or existing client may collect data that is used to further validate and/or train the global machine learning models.

Since the global machine learning models are trained using data from specific microclimates, e.g., specific for the anonymous client, the accuracy of the different discrete model classes and submodel classes of the machine learning models are improved through the sharing of the best parameters that are used for the prediction by the different clients, e.g., has the benefit of developing a global model for each different model class (and submodel class) that is trained from all deployed nodes. While the tuning of the global machine learning models is increased through the sharing of parameters, it is appreciated that since the data that was used to train the machine learning models is kept locally at the node or gateway, e.g., not accessible by the cloud-computing system, the data for the specific client that was used to train the machine learning models and/or any lower layers of the submodel remains anonymous, e.g., any sensitive data or information the client does not want to share is not shared.

The updated global machine learning models may be used by the organization or group to make the necessary recommendations or take the necessary actions based on the predicted soil type, e.g., loam, and soil condition, e.g., dry. For example, when the organization or group is a farmer co-op in a certain region, the co-op may recommend that all farmers having loam that is dry to have an irrigation schedule in which the agricultural crop is irrigated twice a day and fertilized once a month. In another embodiment, an insurance agency may use the updated global machine learning model to predict the soil condition, e.g., wet, to determine the level of insurance to provide to the farmer and what actions should be taken to lower the insurance risk, e.g., crop damage from overly damp soil which causes molds and/or disease. For example, the insurance agency may recommend an aeration schedule of the soil to the farmer to reduce the wetness of the soil, an irrigation schedule, disease mitigation routines, etc.

It is also appreciated that by the new or subsequent client being able to predict the soil type at his or her farm or agricultural site, the prediction may be used for taking additional actions. For example, by knowing the soil type, the soil moisture may be forecasted, because the different soil types hold water longer, e.g., clay holds water longer than sand. Additionally, the prediction for any submodel class, e.g., sandy/clay, would have a water-holding capacity between sand and clay. Thus, the submodel class provides the new or subsequent client finer (or higher) resolution to predict the soil moisture and/or soil matric potential to understand and take action, e.g., by changing or adding irrigation schedules, aeration schedules, disease mitigation routines, etc.

In another exemplary embodiment, the cloud-based computing system may be designed, programmed, or configured to create machine learning models to create a synthetic sensor for agricultural measurements. For example, the synthetic sensor includes a plurality of data feeds from many sensor types or data, e.g., an array of low-cost, lower precision sensors can be used, in which sensor fusion that uses the above machine learning can be used to improve the accuracy of each sensing element by using machine learning to fuse data from the other sensing elements in the array and/or for creating a “synthetic sensor” that replicates the output of high-cost and maintenance intensive sensing devices which is beneficial for agricultural and geophysical science applications. Accordingly, the synthetic sensor allows accurate forecasting of plant stress(es) to provide farmers with the ability to, among other things, confidently irrigate, apply inputs to crops with the precise amount and timing needed to eliminate plant stress, avoid the environmental damage of over application, and increase crop yields while reducing water, fertilizer, and spray applications, and other means for reducing the effect of the plant stress on the plant.

In this embodiment, the problem to be solved is providing synthetic sensors for replicating the performance of an expensive, maintenance prone, or difficult to install sensor, without requiring the presence or continuous presence of that sensor. For example, a synthetic sensor for providing readings for soil moisture, crop yield, soil matric potential, etc. The cloud-based computing system includes a processor, a data storage device, a neural network, and machine readable instructions stored on the data storage device, which when executed by the processor is designed, programmed, or otherwise configured to control the cloud-based computing system to define a plurality of different discrete model classes for the different synthetic sensors. The cloud-based computing system further includes a plurality of datasets that includes different data types associated with the different sensors. For example, the datasets can include air temperature, air humidity, soil tension, recorded precipitation, dew point, UV Index, solar radiation, cloud cover percentage, barometric pressure, a soil temperature, recorded precipitation, dew point, UV Index, solar radiation, cloud cover percentage, VOC, CO2, NO, weather data, or combination thereof. The cloud-based computing system is then configured to use the neural network to train the initial global machine learning models for each discrete model class, e.g., determines the supermodel or seed model to be used by the user client. The plurality of datasets may be divided between a training dataset and a validation training set to test the accuracy of the global machine learning model using transfer learning. It is appreciated that the initial global machine learning model for the discrete model class may include submodel classes related to the discrete model class.

The global machine learning model for each discrete model class and/or any machine learning submodel for the submodel class may then be transmitted and/or downloaded to an anonymous client, e.g., on an operating system of a processor enabled device, for further training and provisioning.

The processor enabled device of the anonymous client may be designed, programmed, or otherwise configured to then collect data for a predetermined amount of time, e.g., 1 month or 30 days. For example, the processor-enabled device of the anonymous client is designed, programmed, or otherwise configured to collect the air temperature, air humidity, soil tension, recorded precipitation, dew point, UV Index, solar radiation, cloud cover percentage, barometric pressure, a soil temperature, recorded precipitation, dew point, UV Index, solar radiation, cloud cover percentage, VOC, CO2, NO, weather data that were found to be feature data, e.g., found to contribute positively to training the global machine learning model, e.g., lower the error. The process-enabled device of the anonymous client may then be designed, programmed, or otherwise configured to process the data collected by the anonymous client and separate the data into a test dataset and a validation dataset. The test dataset and the validation dataset may be separated based on the predetermined amount of time, e.g., when the predetermined amount of time is 1 month, the first three weeks may be used as the test dataset and the last week used as the validation dataset, or other arrangement that can be used for a test dataset and a validation dataset.

Each of the global machine learning models for each discrete model class and/or any submodel class is run using the test dataset to produce a prediction, e.g., in which for N regional soil moisture models for each of the N submodel class, N predictions will be made. The global machine learning models are then validated using the validation dataset to obtain the error for each global machine learning model. Thus, the global machine learning model and/or the submodel that has the lowest error, most-closely describes the regional dialect type for the given anonymous client and is selected and retained for further training. It is appreciated that the remaining less accurate global machine learning models and any submodels may then be discarded.

After selection of the machine learning model with the lowest error, the selected machine learning model is further trained through transfer learning performed locally by the anonymous client. For example, lower layers of the machine learning model are retrained with data collected locally by the anonymous client. The selected global machine learning model is trained until the error of the modelling reaches a predetermined error rate, e.g., between 90%-99% accuracy or 1%-10% error threshold, and preferably between 95-99% accuracy or 1-5% error threshold. Thereby, a local machine learning model may be obtained for the specific regional soil of the at least one anonymous client that is unique to the at least one anonymous client, e.g., based on the soil type. Thus, the trained local machine learning model may be used to predict soil moisture for different regions.

After the local machine learning model reaches the predetermine error rate, the resulting model parameters, e.g., weights, biases, etc., used in the local machine learning models are saved by the processor-enabled device of the anonymous client. The processor enabled device is designed, programmed, or otherwise configured to transmit the parameters of the local machine learning model to the cloud-based computing system periodically, e.g., once a week, once a month, etc. or opportunistically depending on parameter availability, network connectivity, battery state, etc. It is appreciated that other information may also be transmitted to the cloud-based computing system to increase the reliability of the global machine learning models.

Periodically, e.g., once a week, once a month, etc., the cloud-based computing system is designed, programmed, or otherwise configured to update the parameters of the global machine learning models received from the anonymous client(s), e.g., batch model update is performed. In an embodiment, subsequent parameter updates from an anonymous client will overwrite an earlier update. In other embodiments, the parameters are aggregated, averaged, or otherwise computed from a plurality of anonymous clients for the different discrete model classes and machine learning models to maintain anonymity of the client.

The cloud-based computing system may be designed, programmed, or otherwise configured to transmit the updated global machine learning model(s) to new clients and/or existing clients that have the prior versions of the global machine learning model. It is appreciated that the global machine learning model has a high accuracy from initial installation at least because of the use of the transfer learning process for each regional specific machine learning model for each discrete model class and submodel class. That is, a new client or existing client may download the updated global machine learning model that is specific for a region of the new client or existing client, which is more accurate than previous models and does not require the time required to train a site specific model. It is appreciated that the new client and/or existing client may be used for the continued training of the global machine learning models. Specifically, the new client and/or existing client may collect data that is used to further validate and/or train the global machine learning models for each discrete model class and/or submodel class.

In yet another exemplary embodiment, the process for obtaining a trained model privately and securely may be used for predicting text based on regional dialects. Typically, text prediction is provided by passing the previous several words provided by a user through a predictive model to produce a suggestion for the next word. Additionally, suggestions may be provided for misspelled words based on how close the misspelled word is to other words in a given language. It is appreciated that these predictive text models perform better when the models are trained with data that most closely matches the language and dialect of the user.

In this embodiment, the problem to be solved is providing accurate text prediction, where the different discrete model classes may relate to the different languages that are spoken, e.g., English, Spanish, French, etc. For example, a cloud-based computing system, that has similar components as the above embodiments, may be accessed by an organization or group. The cloud-based computing system includes a processor, a data storage device, a neural network, and machine readable instructions stored on the data storage device, which when executed by the processor is designed, programmed, or otherwise configured to control the cloud-based computing system to define a plurality of different discrete model classes for the different languages. The cloud-based computing system further includes a plurality of datasets that includes different words and phrases from the respective model class, e.g., English, French, Spanish, etc. The cloud-based computing system is then configured to use the neural network to train the initial global machine learning models for each model class, e.g., determines the supermodel or seed model to be used by the user client. The plurality of datasets may be divided between a training dataset and a validation training set to test the accuracy of the global machine learning model using transfer learning. It is appreciated that the initial global machine learning model for the model class may include submodel classes related to the model class. For example, the submodel classes for the English model class may include British-English, American-English, Australian-English, etc. and/or further into regional dialects, e.g., Southern, Northeast, Southwest, Midwest, etc.

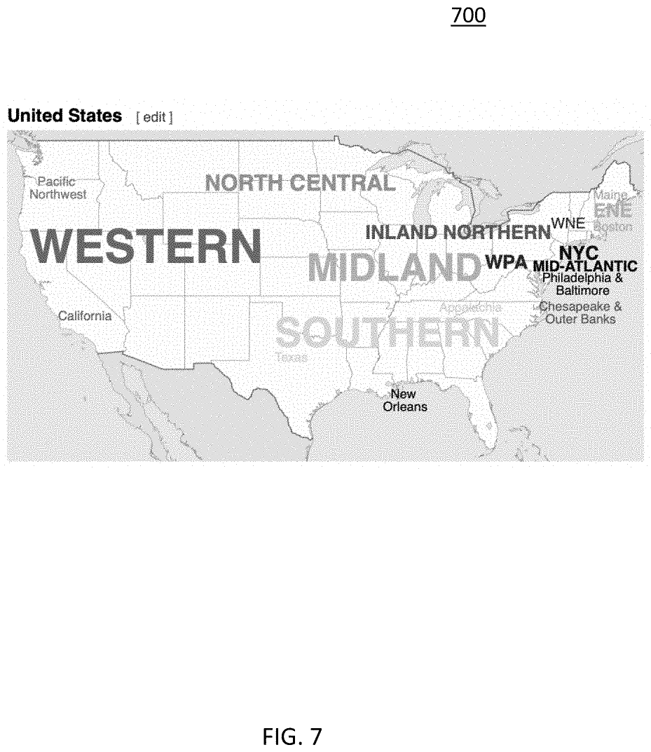

For example, FIG. 7 illustrates an example of the problem to be solved, in which the problem to be solved 700 is determining the submodel classes for the top-level model class of English. The different submodel classes may be, for example, Western, Midland, Southern and North Central. The submodel classes may also include additional sub-submodel classes, for example, Pacific Northwest, Californian, Mid-Atlantic, and further sub-sub-submodel classes, such as Eastern New England, Western New England, New Orleans, Texan, Western Pennsylvanian, New York, etc. and combinations thereof. It is appreciated that the different discrete model classes are now limiting, but provided as examples as how the top-level discrete model classes and submodel classes may be defined.

The global machine learning model for each model class and/or any machine learning submodel for the submodel classes may then be transmitted and/or downloaded to an anonymous client, e.g., on an operating system of a processor enabled device, for further training and provisioning.

The processor enabled device of the anonymous client may be designed, programmed, or otherwise configured to then collect data for a predetermined amount of time, e.g., 1 month or 30 days. For example, the processor-enabled device of the anonymous client is designed, programmed, or otherwise configured to collect the text used by the client and the final text output and/or text correction locally, e.g., at the client site. For example, the processor enabled device may obtain metrics about how often the anonymous client selects or manually types one of the suggested words that are collected. It is appreciated that the predetermined amount of time may be any time length that allows the collection of data found useful for training of the machine learning data, e.g., one day, one week, one month, one year, etc. The process-enabled device of the anonymous client may then be designed, programmed, or otherwise configured to process the data collected by the anonymous client and separate the data into a test dataset and a validation dataset. The test dataset and the validation dataset may be separated based on the predetermined amount of time, e.g., when the predetermined amount of time is 1 month, the first three weeks may be used as the test dataset and the last week used as the validation dataset, or other arrangement that can be used for a test dataset and a validation dataset.

Each of the global machine learning models for each model class and/or any submodel class is run using the test dataset to produce a prediction, e.g., in which for N regional dialect models for each of the N submodel classes, N predictions will be made. The global machine learning models are then validated using the validation dataset to obtain the error for each global machine learning model. For example, if three global machine learning models are used for three discrete model classes, e.g., English, French, Spanish, and the anonymous is in the United States, the global machine learning model for the English model class may be selected. The submodels for the submodel classes for the English model class may also be validated, in which the machine learning models of the submodel classes for the regional dialects, e.g., Northeast, Southwest, Southern, may be validated having an error of 69%, 90% and 98%, respectively. Thus, the global machine learning model and/or the submodel that has the lowest error, most-closely describes the regional dialect type for the given anonymous client and is selected and retained for further training. It is appreciated that the remaining less accurate global machine learning models and any submodels may then be discarded.

After selection of the machine learning model with the lowest error, e.g., the model associated with the South submodel class of the English model class, the selected machine learning model is further trained through transfer learning performed locally by the anonymous client. For example, lower layers of the machine learning model are retrained with data collected locally by the anonymous client, e.g., accuracy of the predictive text. The selected global machine learning model is trained until the error of the modelling reaches a predetermined error rate, e.g., between 90%-99% accuracy or 1%-10% error threshold, and preferably between 95-99% accuracy or 1-5% error threshold. It is also appreciated that the submodel class may further include additional submodel classes, e.g., specific Southern dialects, e.g., New Orleans, Texan, Georgian, etc., related to the selected submodel class. Thereby, a local machine learning model may be obtained for the specific regional dialect of the at least one anonymous client that is unique to the at least one anonymous client. Thus, the trained local machine learning model may be used to predict text (and corrected spellings) for different languages and submodel classes of the languages.

After the local machine learning model reaches the predetermine error rate, the resulting model parameters, e.g., weights, biases, etc., used in the local machine learning models are saved by the processor-enabled device of the anonymous client. The processor enabled device is designed, programmed, or otherwise configured to transmit the parameters of the local machine learning model to the cloud-based computing system periodically, e.g., once a week, once a month, etc. or opportunistically depending on parameter availability, network connectivity, battery state, etc. It is appreciated that other information may also be transmitted to the cloud-based computing system to increase the reliability of the global machine learning models. For example, the model class that was selected, e.g., Southern and New Orleans submodel class, may be sent to the cloud-based computing system and/or any information that the anonymous client determines would be useful for the global machine learning model but which still maintains the anonymity of the anonymous client, e.g., a region, state, country of the anonymous client and not specific details of the anonymous client such as, age, gender, address, etc.

Periodically, e.g., once a week, once a month, etc., the cloud-based computing system is designed, programmed, or otherwise configured to update the parameters of the global machine learning models received from the anonymous client(s), e.g., batch model update is performed. In an embodiment, subsequent parameter updates from an anonymous client will overwrite an earlier update. In other embodiments, the parameters are aggregated, averaged, or otherwise computed from a plurality of anonymous clients for the different discrete model classes and machine learning models to maintain anonymity of the client.

In an embodiment, the cloud-based computing system is designed, programmed, or otherwise configured to output a global machine learning model for a model class (or submodel class) only after a predetermined amount of parameter updates have been received. For example, after each of the parameters of the global machine learning model have received two parameter updates, preferably five parameters updates, and most preferably ten parameter updates, the cloud-based computing system will transmit a given predictive text model to a user client. The updated global machine learning model may replace the prior version(s) of the global machine learning model or be used to aggregate the parameters of the global machine learning model. It is appreciated that the machine learning models for the any of the submodel classes may be used to update the global machine learning model, e.g., top-level models, for the respective model class. For example, the parameters from the submodel class Southern, may be aggregated and used to update the parameters for the machine learning model for the English model class, e.g., the top-level supermodel.

The cloud-based computing system may be designed, programmed, or otherwise configured to transmit the updated global machine learning model(s) to new clients and/or existing clients that have the prior versions of the global machine learning model. It is appreciated that the global machine learning model has a high accuracy from initial installation at least because of the use of the transfer learning process for each regional dialect specific machine learning model for each model class and submodel class. That is, a new client or existing client may download the updated global machine learning model that is specific for a regional dialect of the new client or existing client, which is more accurate than previous models and does not require the time required to train a site specific model. It is appreciated that the new client and/or existing client may be used for the continued training of the global machine learning models. Specifically, the new client and/or existing client may collect data that is used to further validate and/or train the global machine learning models for each model class and/or submodel class.

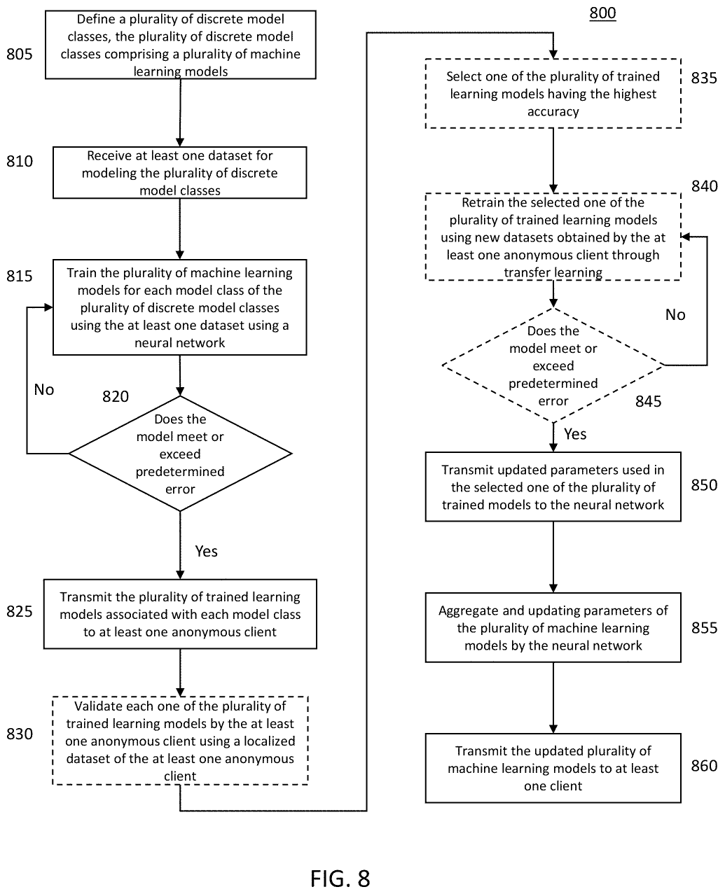

FIG. 8 illustrates an exemplary work flow 800 for obtaining a trained model privately and securely, according to at least one example embodiment described herein.

As shown in FIG. 8, Block 805 represents the initial defining of the plurality of discrete model classes for the problem to be solved. The plurality of discrete model classes includes a plurality of machine learning models that model the prediction for the problem to be solved based in data received at the input. The problem to be solved may be a problem defined by an organization or group that may relate to the same particular problem, relate to different problems to be solved, and/or combinations thereof. For example, the problem may be determining the soil type for a particular client. Block 805 may be followed by Block 810.

In Block 810, the data for the input for the plurality of machine learning models are received as a dataset, in which the dataset may include data that that has been collected and/or provided that may directly or indirectly influence the particular problems identified for the plurality of discrete model classes. For example, the dataset includes non-anonymous datasets that may be general/public data, provided by certain clients, groups of clients, organizations, etc., or any combination thereof related to the particular problem to be solved for the plurality of discrete model classes. The dataset may include data, for example, related to general types of soil, e.g., clay, sand, silt, loam, combinations thereof, etc. and different conditions, e.g., wet, dry, very dry, very wet, ideal, cold, hot, etc. For example, the datasets may include soil temperature data, soil volumetric data, soil matric potential data, soil wetness data, soil density data, air humidity sensor, air temperature sensor, a barometric pressure, recorded precipitation, dew point, UV Index, solar radiation, cloud cover percentage, etc. Block 810 may be followed by Block 815.

In at least one example embodiment, in Block 815 the plurality of machine learning models is trained for each model class based on the dataset using a neural network. The plurality of machine learning models includes machine learning models for each model class of the plurality of discrete model classes, e.g., creates global models for each top-level model class, and may further include machine learning models for any submodel classes until all or most discrete model classes and/or submodel classes are defined by a machine learning model. For example, in an embodiment, a machine learning model, e.g., a top-level model, may be provided for each model class, e.g., type of soil condition, etc., silt, sand, clay, loam, and combinations thereof. Lower-level submodels may be provided for each of the submodel classes, for example, machine learning models for silt in wet conditions, silt in dry conditions, etc. Thus, the trained machine learning model outputs the type of soil and/or soil condition from the inputs provided from the dataset. Block 815 may be followed by Block 820.

Block 820 is a decision block to determine whether or not the machine learning model is trained. The modeling to the datasets may be considered complete and be used as a machine learning model, when the error of the modeling reaches a predetermined error rate, e.g., between 80%-99% accuracy or 1%-20% error threshold, and preferably between 90-95% accuracy or 5-10% error threshold. Error is calculated by comparing the model's accuracy against a known target dataset, with lower error being better. To make the model broadly applicable and not overfitted for one particular dataset, transfer learning may be used to adapt a model previously trained on a single dataset to generalize across several disparate datasets that can be unique to specific a geographic location, soil type, ambient environment, etc. If the modeling does not have the required error threshold, the modeling is continued until the machine learning model meets the error threshold. Block 820 may be followed by Block 825.

In Block 825, the plurality of trained learning model associated with each model class are transmitted to at least one anonymous client for further training and provisioning. Optionally, Block 825 may be followed by Block 830, in which the anonymous client runs and validates each of the plurality of machine learning models for each model class of the plurality of discrete model classes using data provided locally at the at least one anonymous client, e.g., local database. It is appreciated that the local databases may also include encrypted databases, e.g., AWS. That is, the local databases are datasets that are locally controlled by the anonymous client and not otherwise accessible by a third-party.

The data is collected for a predetermined amount of time, e.g., 1 month or 30 days. It is appreciated that the predetermined amount of time may be any time length that allows the collection of data for training the machine learning model, e.g., one day, one week, one month, one year, etc. The anonymous client may then separate the collected data into a test dataset and a validation dataset. The test dataset and the validation dataset may be separated based on the predetermined amount of time, e.g., when the predetermined amount of time is 1 month, the first three weeks may be used as the test dataset and the last week used as the validation dataset, or other arrangement that can be used for establishing a test dataset and a validation dataset.

Each of the machine learning models for each model class (or submodel class) is run using the test dataset to produce a prediction. The machine learning models are then validated using the validation dataset to obtain the error for each machine learning model. Block 830 may optionally be followed by Block 835.

In Block 835, after all of the plurality of machine learning models are run by the anonymous client, the machine learning model having the highest accuracy among the plurality of machine learning models based on the local dataset is selected, e.g., a machine learning model that has between 80-95% accuracy. For example, if three machine learning models are used for three discrete model classes of soil types, e.g., clay, silt, and loam, the error may be 69%, 90% and 98%, respectively, for the three different discrete model classes of soil types. Thus, the machine learning model that has the lowest error, most-closely describes the soil type at the given anonymous client site location and is selected and retained for further training. It is appreciated that the remaining less accurate global machine learning models may then be discarded and/or deleted, e.g., removed by the at least one anonymous client 140. Block 835 may be followed by Block 840.

In Block 840, after selection of the machine learning model with the lowest error, the anonymous client continues using the selected machine learning model to provide predictions and the selected machine learning model is retrained using data collected locally by the anonymous client, e.g., using transfer learning, in which lower layers in the machine learning model are retrained with the local data in which the local data that is collected is separated as a test dataset and validation dataset. Optionally, in Block 845, the selected machine learning model is trained until the error of the modelling reaches a predetermined error rate, e.g., between 90%-99% accuracy or 1%-10% error threshold, and preferably between 95-99% accuracy or 1-5% error threshold. Error is calculated by comparing the model's accuracy against the local dataset, with lower error being better. Block 840 or 845 may then be followed by Block 850.

In Block 850, the resulting model parameters, e.g., weights, biases, etc., used in the local machine learning models are saved by the at least one anonymous client and the parameters of the local machine learning model may be transmitted or uploaded to the cloud-based computing system periodically, e.g., once a week, once a month, etc. or opportunistically depending on parameter availability, network connectivity, battery state, etc. It is appreciated that other information may also be transmitted to the cloud-based computing system to increase the reliability of the machine learning models. Block 850 may be followed by Block 855.

In Block 855, the neural network (or the cloud-based computing system) aggregates and updates the parameters of the plurality of machine learning models for the respective model class(ies). Periodically, e.g., once a week, once a month, etc., the cloud-based computing system may update the parameters of the machine learning models received from the anonymous client(s), e.g., batch model update is performed in which the plurality of trained learning models is retransmitted to at least one anonymous client and the anonymous client repeats the validation, selection, training, and transmission of updated parameters of the selected machine learning model. In an embodiment, subsequent parameter updates from an anonymous client will overwrite an earlier update. In other embodiments, the parameters are aggregated, averaged, weighted, or otherwise computed from a plurality of anonymous clients for the different discrete model classes and machine learning models to maintain anonymity of the client. Block 855 may be followed by Block 860.

In Block 860, the updated machine learning models may be transmitted to at least one client. That is, over time, the RNN can incorporate continuous transfer learning to tune to specific geographical locations of various clients to improve the accuracy of predictions. Thus, the global machine learning model for the respective model class, e.g., soil type silt, is improved for subsequent clients by using anonymous data that closest matches the subsequent client to obtain the most relevant parameters for the machine learning model. It is appreciated that each of the global machine learning models may then be updated in similar manners using anonymous clients based on localized datasets. Since only the parameters of the models are transmitted by the at least one anonymous client and the parameters are aggregated by the cloud-based computing system, anonymity of the dataset is preserved.

While the foregoing description has been provided with the advantages as discussed herein, it is appreciated that other advantages are also provided. For example, since the processor enabled device of the clients are only designed, programmed, or otherwise configured to only upload the parameters and/or weights of the trained machine learning models, which have a much smaller data size than the machine learning model(s) and/or the local data themselves, less internet bandwidth is required for communicating/transmitting the parameters and/or weights to the cloud-based computing system. Additionally, since the global machine learning models for each model class (and/or submodel class) are trained and improved by the anonymous clients, the specific machine learning model that best fits the environment for the new or subsequent client may be preinstalled or downloaded on the site specific device, e.g., on the device firmware on the sensor, in cases where limited or no internet connectivity is available, e.g., remote locations, and be able to provide accurate predictions upon installation.

The foregoing description is presented to enable one of ordinary skill in the art to make and use the disclosed embodiments and modifications thereof, and is provided in the context of a patent application and its requirements. Various modifications to the disclosed embodiments and the principles and features described herein will be readily apparent to those of ordinary skill in the art. For example, the different features in the description for the system and method may be combined or interchanged accordingly. Thus, the present disclosure is not intended to limit the invention to the embodiments shown; rather, the invention is to be accorded the widest scope consistent with the principles and features described herein.

What is claimed is:

1. A computing system for obtaining a trained model privately and securely, the system comprising:
at least one processor;
at least one data storage device;
a neural network; and
machine readable instructions stored in the at least one data storage device that when executed by the at least one processor controls the system to:
define, in a cloud-based computing system, a plurality of discrete model classes, wherein the plurality of discrete model classes comprises a plurality of machine learning models;
receive by the cloud-based computing system, at least one dataset for modeling the plurality of discrete model classes;
train at least one respective machine learning model of the plurality of machine learning models for each discrete model class of the plurality of discrete model classes using the at least one dataset using the neural network;
transmit the plurality of trained learning models associated with each discrete model class to at least one anonymous client;
receive updated parameters from the at least one anonymous client, wherein the updated parameters are from a selected one of the plurality of trained models by the at least one anonymous client;
aggregate and update parameters of the plurality of machine learning models by the neural network;
transmit the updated plurality of machine learning models to at least one client.

2. The computing system according to claim 1, wherein the at least one anonymous client comprises a processor enabled device comprising memory, a processor, and machine readable instructions stored in the memory that when executed by the processor controls the processor enabled device to:
validate each one of the plurality of trained learning models using a localized dataset,
select one of the plurality of trained learning models having the highest accuracy among the plurality of trained learning models,
retrain the selected one of the plurality of trained learning models using new datasets obtained by the at least one anonymous client through transfer learning.

3. The computing system according to claim 1, wherein the plurality of discrete model classes is subdivided into a plurality of submodel classes, wherein each submodel class of the plurality of submodel classes comprises at least one machine learning model.

4. The computing system according to claim 3, further comprising transmitting the at least one machine learning model from any submodel class of the plurality of submodel classes associated with the selected one of the plurality of trained learning models having the highest accuracy.

5. The computing system according to claim 3, wherein the processor enabled device is further configured to:
validate each one of the at least one machine learning model for the submodel class using a localized dataset of the at least one anonymous client;
select one of the plurality of trained learning models from the submodel class having the highest accuracy;
retrain the selected one of the plurality of trained learning models from the submodel class using new datasets obtained by the at least one anonymous client through transfer learning;
transmit updated parameters used in the selected one of the plurality of trained models of the submodel class to the neural network.

6. The computing system according to claim 2, wherein the processor enabled device is further configured to delete any remaining trained learning model that was not selected as having the highest accuracy.

7. The computing system according to claim 2, wherein the system is further configured to:
retransmit the plurality of trained learning models associated with each discrete model class to the at least one anonymous client after a predetermined amount of time; and
the at least one anonymous client is further configured to:
validate each one of the plurality of trained learning models using the localized dataset of the at least one anonymous client;
select one of the plurality of trained learning models having the highest accuracy;
train the selected one of the plurality of trained learning models using new datasets obtained by the at least one anonymous client through transfer learning;
transmit the updated parameters used in the selected one of the plurality of trained models to the neural network.

8. The computing system according to claim 7, wherein the predetermined amount of time is every thirty days or monthly.

9. The computing system according to claim 1, wherein the at least one anonymous client is configured to transmit any updated parameters used in the selected one of the plurality of trained models to the cloud-based computing system after a predetermined amount of time.

10. The computing system according to claim 1, wherein the plurality of discrete model classes is directed to soil mapping and the at least one dataset includes data from representative soil types.

11. The computing system according to claim 10, wherein the plurality of machine learning models comprises machine learning models for each representative soil type.

12. The computing system according to claim 10,
wherein the localized dataset comprises data obtained from a plurality of sensors installed at a location of the at least one anonymous client, and
wherein the plurality of sensors collect data that includes at least one of soil volumetric moisture, soil capacitance, soil tension, soil temperature, air humidity, air temperature, and barometric pressure.

13. The computing system according to claim 11, further comprising:
downloading a soil-specific machine learning model from the updated plurality of machine learning models for a soil type of the at least one client, and
wherein the updated plurality of machine learning models are soil-specific models for each discrete model class of the plurality of discrete model classes.

14. The computing system according to claim 1, wherein the plurality of discrete model classes is directed to predictive text for regional dialects and the at least one dataset includes data from different regions that speak the dialect.

15. A method for obtaining a trained model privately and securely, the method comprising:
defining, in a cloud-based computing system, a plurality of discrete model classes, the plurality of discrete model classes comprising a plurality of machine learning models;
receiving, by the cloud-based computing system, at least one dataset for modeling the plurality of discrete model classes;
training the plurality of machine learning models for each discrete model class of the plurality of discrete model classes using the at least one dataset using a neural network;
transmitting the plurality of trained learning models associated with each discrete model class to at least one anonymous client;
validating each one of the plurality of trained learning models by the at least one anonymous client using a localized dataset of the at least one anonymous client;
selecting one of the plurality of trained learning models having the highest accuracy;
retraining the selected one of the plurality of trained learning models using new datasets obtained by the at least one anonymous client through transfer learning;
transmitting updated parameters used in the selected one of the plurality of trained models to the neural network;
aggregating and updating parameters of the plurality of machine learning models by the neural network; and
transmitting the updated plurality of machine learning models to at least one client.
